(19) United States
(12) Patent Application Publication
(10) Pub. No.: US 2024/0102114 A1
(43) Pub. Date: Mar. 28, 2024

(54) PROXIMITY LIGATION ASSAY (PLA)-BASED DETECTION METHOD FOR HIGH-ORDER STRUCTURE (HOS) OF RNA VIRUS

(71) Applicant: ACADEMY OF MILITARY MEDICAL SCIENCES OF ACADEMY OF MILITARY SCIENCES PLA CHINA, Beijing (CN)

(72) Inventors: Yan ZHANG, Beijing (CN); Zhihu ZHAO, Beijing (CN); Wenlong SHEN, Beijing (CN); Ping LI, Beijing (CN); Shu SHI, Beijing (CN)

(73) Assignee: ACADEMY OF MILITARY MEDICAL SCIENCES, Beijing (CN)

(21) Appl. No.: 17/774,495

(22) PCT Filed: May 28, 2021
(86) PCT No.: PCT/CN2021/096748

§ 371 Date: May 5, 2022

(30) Foreign Application Priority Data
Apr. 25, 2021 (CN) 202110447273.0

(51) Int. Cl. C12Q 1/70; C12Q 1/6806

(52) U.S. Cl. CPC C12Q 1/701; C12Q 1/6806

(57) ABSTRACT

Provided is a proximity ligation assay-based detection method for a high-order structure (HOS) of an RNA virus, including: mixing an RNA virus with a cross-linking agent, conducting cross-linking under ultraviolet (UV) light, and recovering the RNA virus to obtain a cross-linked RNA virus; extracting RNA of the cross-linked RNA virus; conducting fragmentation on the RNA with RNase III to obtain RNA fragments; ligating the RNA fragments and conducting decrosslinking to obtain decrosslinked RNA fragments; constructing a sequencing library for the decrosslinked RNA fragments; and conducting high-throughput sequencing on the sequencing library, and conducting an RNA HOS analysis on a sequencing result. In the present disclosure, high-efficiency short-distance ligation reaction is used to realize the cross-linking of RNA in virus particles in cell culture or collected supernatant, so as to analyze the HOS of RNA virus genome.

### CROSS REFERENCE TO RELATED APPLICATION

This patent application is a national stage entry filing under 35 USC 371 to PCT/CN2021/096748 filed on May 28, 2021, which claims the benefit and priority of Chinese Patent Application No. 2021104472730, filed with the China National Intellectual Property Administration (CNIPA) on Apr. 25, 2021, entitled “PROXIMITY LIGATION ASSAY (PLA)-BASED DETECTION METHOD FOR HIGH-ORDER STRUCTURE (HOS) OF RNA VIRUS”, the disclosure of which is incorporated by reference herein in its entirety as part of the present application.

### TECHNICAL FIELD

The present disclosure relates to the technical field of virus detection, and particularly relates to a proximity ligation assay (PLA)-based detection method for a high-order structure (HOS) of an RNA virus.

### BACKGROUND ART

Virus is the simplest living organism found so far. Except for prions, the virus is composed of nucleic acids and proteins. Viruses can be divided into RNA viruses and DNA viruses according to the type of nucleic acid. A complete viral nucleic acid is also called a viral genome. The viral genome is a full set of genetic codes of the virus, and the viral genome guides the encoding of all viral proteins and regulates the life cycle of the virus. Recent studies have shown that the viral genome has the function of encoding viral proteins, and fragments of the viral genome can be folded with each other to form a complex spatial structure. This spatial structure (HOS) has great significance for gene coding and the infection and replication of the virus. Therefore, it is of great significance to study the HOS of the viral genome to understand the pathogenicity and the infection and replication of the virus.

Theoretically, identification methods of the HOS of RNA or DNA in cells are suitable for studying the structure of the viral genome. At present, the techniques for studying the HOS of the RNA in the cells can be roughly divided into the following categories: X-ray, nuclear magnetic resonance (NMR), click chemistry and existing PLA. Both the X-ray and the NMR involve high resolution, but have deficiencies of complex technology and inability to study the RNA structures under physiological conditions. These two methods are suitable for fine determination of the structure of RNA complexes. The click chemistry features simplicity and high-throughput, but can only determine whether the RNA is double-stranded and cannot determine the interaction. This method is suitable for the prediction of intracellular RNA structures. The existing PLA is suitable for intracellular RNA structures and interaction, and also has high throughput and RNA structure mapping under physiological conditions. However, this method has complicated operation steps and high requirements for samples, which is not suitable for studying low-level virus samples.

The foundation of studying the HOS of RNA is to study the frequency of spatial contact or interaction between local fragments in RNA molecules. To solve the above problems, researchers have developed a series of research techniques in recent years. A basic idea of these techniques is to fixate RNAs close to each other (by interaction) using an RNA cross-linking agent, treat RNA terminals and ligate interacted RNA fragments, and identify the occurrence frequency of “chimeric” RNAs through high-throughput sequencing and bioinformatics analysis, thereby determining the interaction of RNA fragments. These research methods have played an important role in identifying RNA structures and interactions under different physiological conditions since early invention. All methods in papers published in the earlh stage included the enrichment of cross-linked fragments, such that high requirements on the initial sample size are raised. Generally, at least 20 μg of total RNA was required to meet the experimental needs. However, compared with intracellular RNA, the viral genome has the following characteristics: the viral genome may have a low number of copies and a very low total amount of viral nucleic acid; and the viral genome accounts for a very low number of all host genes. Therefore, the conventional research strategy of RNA structure has many difficulties in studying the structure of the viral genome. In particular, it is difficult to meet the amount of viral nucleic acids required for the experiment, resulting in insufficient analysis coverage, which in turn leads to the loss of a large number of structural details.

### SUMMARY

In view of this, the purpose of the present disclosure is to provide a PLA-based detection method for a high-order structure (HOS) of an RNA virus. The method can analyze the HOS of the RNA viral genome on low-concentration virus samples, and relatively comprehensive HOS information can be obtained.

The present disclosure provides a PLA-based method for detecting HOS of an RNA virus, which includes the following steps:

1) mixing an RNA virus with a cross-linking agent, conducting cross-linking under ultraviolet (UV) light, and recovering the RNA virus to obtain a cross-linked RNA virus;

2) extracting RNA of the cross-linked RNA virus in step 1);

3) conducting fragmentation on the RNA in step 2) with an RNase III to obtain RNA fragments;

4) ligating the RNA fragments in step 3) and decrosslinking the RNA fragments to obtain decrosslinked RNA fragments;

5) constructing a sequencing library for the decrosslinked RNA fragments in step 4); and

6) conducting high-throughput sequencing on the sequencing library in step 5), and conducting RNA HOS analysis on a sequencing result.

Preferably, the cross-linking agent in step 1) may be a phosphate-buffered saline (PBS) solution containing a psoralen-derived cross-linking agent; and

the psoralen-derived cross-linking agent may have a final concentration of 1-4 μmol/L.

Preferably, the psoralen-based cross-linking agent may include 4′-aminomethyl-4,5′,8-trimethylpsoralen (AMT) or EZ-Link™ Psoralen-PEG3-Biotin.

Preferably, the cross-linking agent may further include digitonin with a mass concentration of 0.01-1%.

Preferably, the RNA virus may have a final concentration of 10⁷-10⁹ copies/mL after the mixing in step 1).

Preferably, in step 1), the UV light may have a wavelength of 360-370 nm; and

the cross-linking may last for 15-25 min.

Preferably, a reaction system for the fragmentation with an RNase III in step 3) may include 1 μl of 10×RNase III buffer, 200 ng of RNA and 1 μl of RNase III, supplemented to 20 μl with RNase-free water.

Preferably, the fragmentation with an RNase III may be conducted for 1-10 min at 36-38° C.

Preferably, in step 4), the decrosslinking may be conducted by irradiating the RNA fragments with the UV light;

the UV light may have a wavelength of 250-260 nm; and

the irradiating may last for 1-10 min.

Preferably, the RNA virus may include a coronavirus and a Coxsackie virus.

The present disclosure also provides a construction method of a sequencing library for proximity ligation assay (PLA)-based detection of a high-order structure (HOS) of an RNA virus, including the following steps:

1) mixing an RNA virus with a cross-linking agent, conducting cross-linking under ultraviolet (UV) light, and recovering the RNA virus to obtain a cross-linked RNA virus;

2) extracting RNA of the cross-linked RNA virus in step 1);

3) conducting fragmentation on the RNA extracted in step 2) with RNase III to obtain RNA fragments;

4) ligating the RNA fragments in step 3) and conducting decrosslinking to obtain decrosslinked RNA fragments; and

5) constructing a sequencing library for the decrosslinked RNA fragments in step 4).

Preferably, the cross-linking agent in step 1) is a phosphate-buffered saline (PBS) solution containing a psoralen-derived cross-linking agent; and

the psoralen-derived cross-linking agent has a final concentration of 1-4 μmol/L.

Preferably, the psoralen-based cross-linking agent includes 4′-aminomethyl-4,5′,8-trimethylpsoralen (AMT) or EZ-Link™ Psoralen-PEG3-Biotin.

Preferably, the cross-linking agent further includes digitonin with a mass concentration of 0.01-1%.

Preferably, the RNA virus has a final concentration of 10⁷-10⁹ copies/mL after the mixing in step 1).

Preferably, in step 1), the UV light has a wavelength of 360-370 nm; and

the cross-linking lasts for 15-25 min.

Preferably, a reaction system for the fragmentation with RNase III in step 3) includes 1 μl of 10×RNase III buffer, 200 ng of RNA and 1 μl of RNase III, supplementing to 20 μl with RNase-free water.

Preferably, the fragmentation with RNase III is conducted for 1-10 min at 36-38° C.

Preferably, in step 4), the decrosslinking is conducted by irradiating the RNA fragments with the UV light;

the UV light has a wavelength of 250-260 nm; and

the irradiating lasts for 1-10 min.

Preferably, the RNA virus includes a coronavirus and a Coxsackie virus.

The present disclosure provides a PLA-based method for detecting HOS of an RNA virus. In the present disclosure, the RNA virus is cross-linked by UV light under the action of a cross-linking agent, such that the interacted (close) RNA fragments form a covalent bond; on the basis of a relatively low initial sample size, fragmentation is conducted using RNase III nuclease to ensure that each fragmented RNA end is suitable for ligation, which is beneficial to improve the ligation efficiency. The method can also help simplify the operation and reduce the loss of RNA fragments, so that the detection of HOS of RNA viruses is also suitable for low-concentration virus samples. The method provided by the present disclosure is called a high-throughput RNA interaction analysis (Hi-R) method. The Hi-R can map in vivo paired RNA interactions with high sensitivity across the whole genome. In addition, the method provided by the present disclosure can reduce the loss of RNA due to terminal treatment and enrichment of chimeric fragments, thereby making the RNA suitable for direct experiments on small amounts of virus particles. The Hi-R method provided by the present disclosure can be used to map the interaction of fragments and HOS maps in the viral genome, providing a basis for studying structural changes in the life cycle of related viruses and its relationship with biological functions.

### BRIEF DESCRIPTION OF THE DRAWINGS

FIG. 1 is a detection of RNA viruses using the Hi-R method provided by the present disclosure. FIG. 1A is a schematic diagram of sample collection and main experimental steps; where virus-infected cells and culture supernatants are collected at different stages of virus infection, and a psoralen-derived cross-linking agent is added to fixate interacted RNA fragments; the RNA is fragmented and ligated, and a cDNA library is established for chimeric RNA fragments formed by the ligation, and high-throughput sequencing is conducted. FIG. 1B is a Dotplot display of chimeric read counts from two replicates, indicating that the proximity scheme has desirable reproducibility. FIG. 1C is a heat map of RNA-RNA interaction of a SARS-CoV-2 virus, where each point represents an interaction signal between genome coordinates on x and y axes, the X axis represents a coordinate of a 5′-arm of a chimera, and the Y axis represents a 3′-arm of the chimera, such that a 5′-3′ chimera is above a diagonal, and a 3′-5′ chimera is below the diagonal. FIG. 1D is statistical data of a single-terminal RNA, the 3′-5′ chimera and the 5′-3′ chimera mapped in each sample.

FIG. 2 is identification of a variable untranslated region (UTR) structure of COVID-19 using the Hi-R method. FIG. 2A is a standardized contact matrix in a 5′-UTR area. FIG. 2B is a standardized SARS-CoV-2 5′-UTR structure, where the color represents a log 2 chimeric read count that supports non-redundant chimeric reads per base pair. FIG. 2C is a standardized contact matrix in a 3′-UTR area. FIG. 2D is a standard SARS-CoV-2 3′-UTR and a variable S2M structure, where base pairing of an arch is specified, and the color represents a log 2 chimeric read count that supports non-redundant chimeric reads per base pair. FIG. 2E is a standardized contact matrix that supports genome cyclization. FIG. 2F shows the base pairings of the 5′-UTR and the 3′-UTR in C, L and V samples, where the color represents a log 2 chimeric read count that supports non-redundant chimeric reads per base pair.

FIG. 3 shows a remote interaction of a TRS-L seat and a TRS-B seat discovered by the Hi-R method. FIG. 3A shows a binding position of a TRS-L region (the first 100 nt) along a SARS-CoV-2 genome in a designated sample, where the 3′-5′ chimera and the 5′-3′ chimera are drawn, respectively, and a black arrow indicates other peaks in orfla. FIG. 3B shows rich interaction peaks of the TRS-L derived from a Z-scoring method, where chimeric read counts from bin-bin contacts are normalized by a Z-score, and TRS-L-mediated interactions with a Z-score>2.13 (a 95% confidence level above the average) is mapped. FIG. 3C shows the distribution of binding sites in the TRS-L region (the first 100 nt), where chimeras that break at a completely specific base are counted, indicating that a ligation occurs at different sites. FIG. 3D shows a contact matrix of a 3′-5′ chimeric reading code across a TRS-L:S binding site, where the color indicates the number of chimeric reads per 1 million mapped reads (also known as counts per million, CPM). FIG. 3E shows the specific site of chimeric reads mapping that supports interaction of TRS-L and S gene. Each line represents a mapping of a read. From this figure, the details of each chimeric read that supports the interaction of TRS-L and S gene can be reflected. It is found that these interactions may come from two modes of sgRNA circularization and TRS-L interaction. FIG. 3F shows the details of base complementation of interaction fragments of the TRS-L and S gene found based on the above analysis.

FIG. 4 is comparison results of structures of a virus in different states. FIG. 4A is a heat map showing the comparison of RNA-RNA interaction in virus particles with cells of the early stage of infection (VvsC) and in virus particles and cell lysates of the late stage of infection (VvsL), where the VvsL is in the upper quadrant and the VvsC is in the lower quadrant. FIG. 4B shows the span distribution of the interaction with different intensities, where a dot plot shows a distribution of differential interaction, *** p<0.001, and two-way and two-sample Kolmogorov-Smirnov test. FIG. 4C shows that the structural domain characteristics are maintained during the life cycle of the SARS-CoV-2 virus; where a heat map shows the normalized average interaction frequency of all boundaries and their vicinity (±0.5 domain length) in the C, L and V samples, and the heat map is divided into windows with a resolution of 10 nt. FIG. 4D maps an average normalized insulation score around the boundary from ½ upstream to ½ downstream. FIG. 4E is a violin diagram comparing the boundary strength of the C, L and V samples, showing a higher boundary strength in the V sample. FIG. 4F is an RNA interaction map (top) aliquoted with a resolution of 10 nt, showing that an interaction distance on the SARS-CoV-2 genome in the C, L and V samples is 10-15 kb, where a line graph (median) shows an insulation curve, and a short line (bottom) reflects the boundaries.

FIG. 5 shows results of contact matrix comparing two biological replicates, where a Coxsackie virus particle RNA of the two biological replicates is processed by Hi-R experiment, a contact matrix diagram shows that the biological replicates have high similarity.

FIG. 6 shows the comparison result of Coxsackie virus structure before and after GFP insertion. FIG. 6A is a heat map of RNA-RNA interaction of a Coxsackie virus CVB3 type. FIG. 6B is a heat map of RNA-RNA interaction of the Coxsackie virus CVB3 type after GFP insertion. FIG. 6C is a difference map of interaction before and after GFP insertion, where red dots represent enhanced interactions after GFP insertion, and blue dots represent weakened interactions after GFP insertion.

FIG. 7 is the results of comparing structural characteristics of two Coxsackie viruses. FIG. 7A shows the characteristics of a Coxsackie viral genome domain before and after GFP insertion described using orientation index, showing that the domain is enhanced after GFP insertion. FIG. 7B shows the characteristics of the Coxsackie viral genome domain before and after GFP insertion described using intensity index, showing that the domain is enhanced after GFP insertion.

FIG. 8 shows the test results of cross-linking efficiency of the Coxsackie virus RNA.

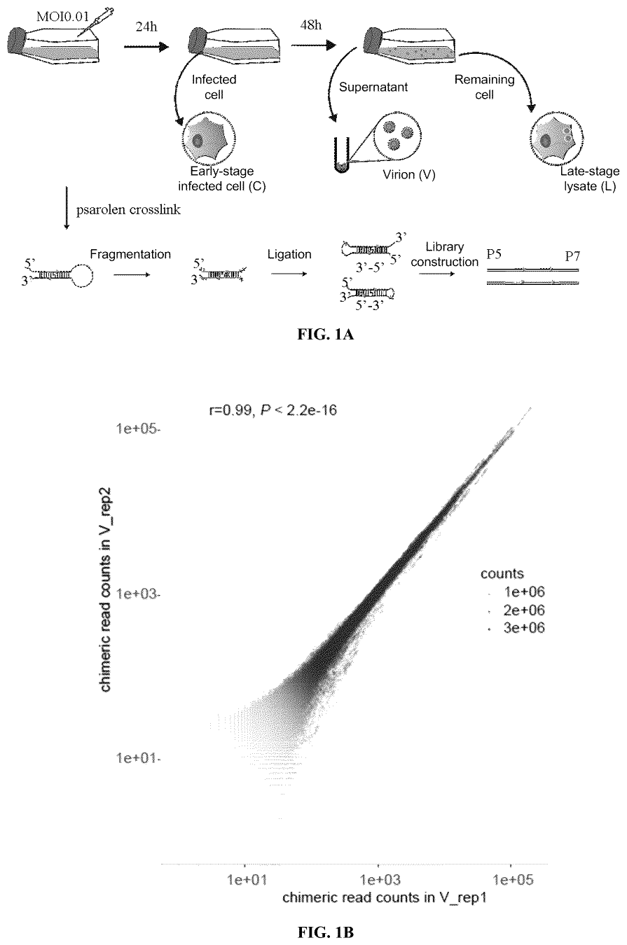

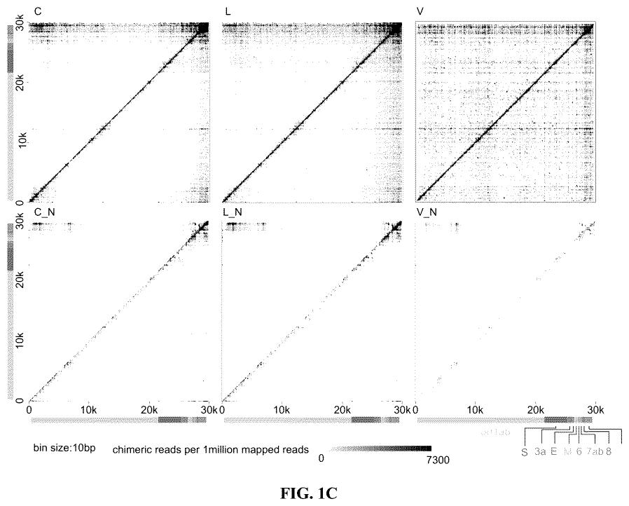

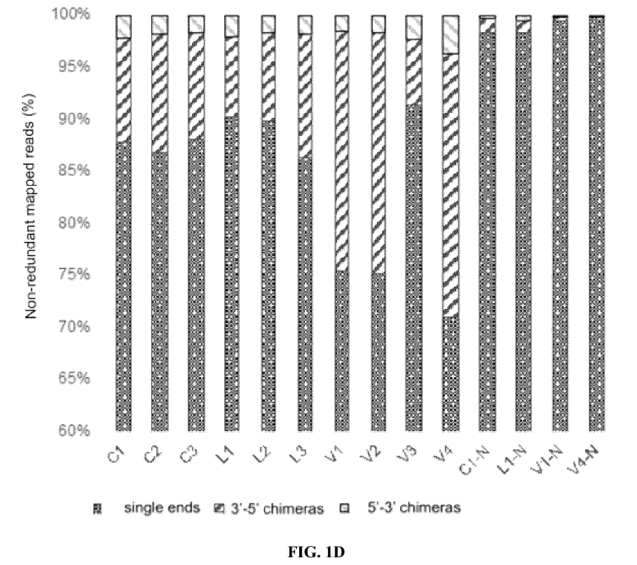

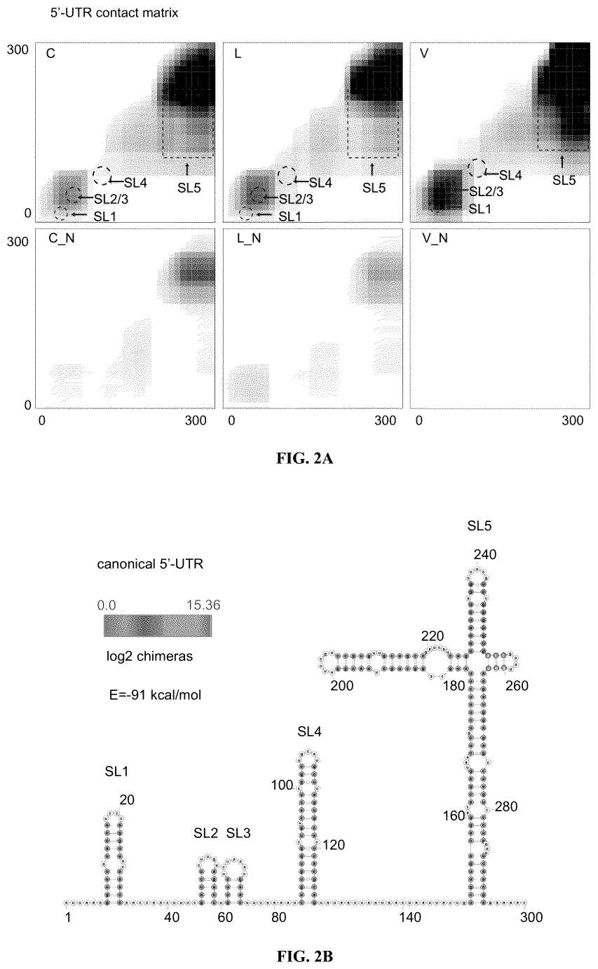

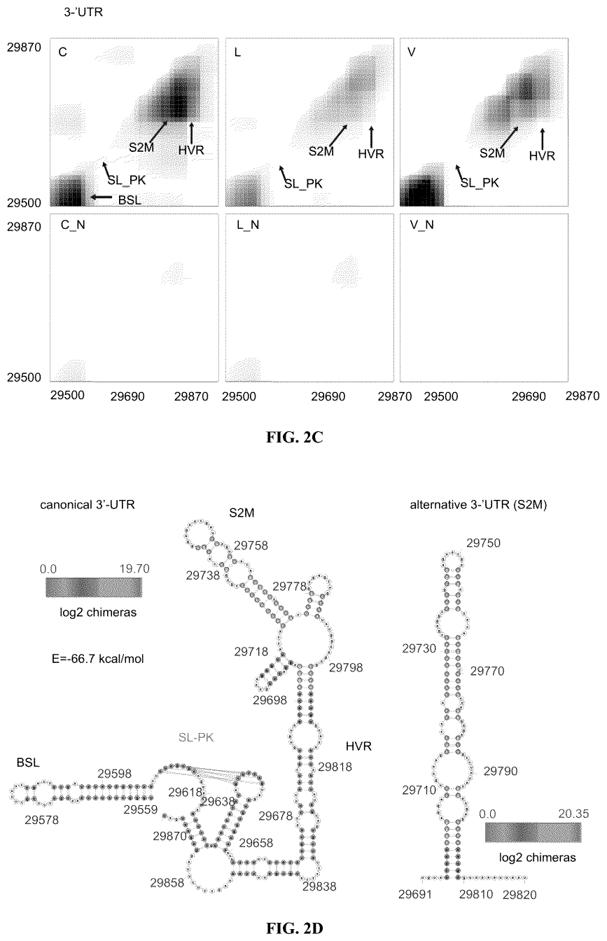

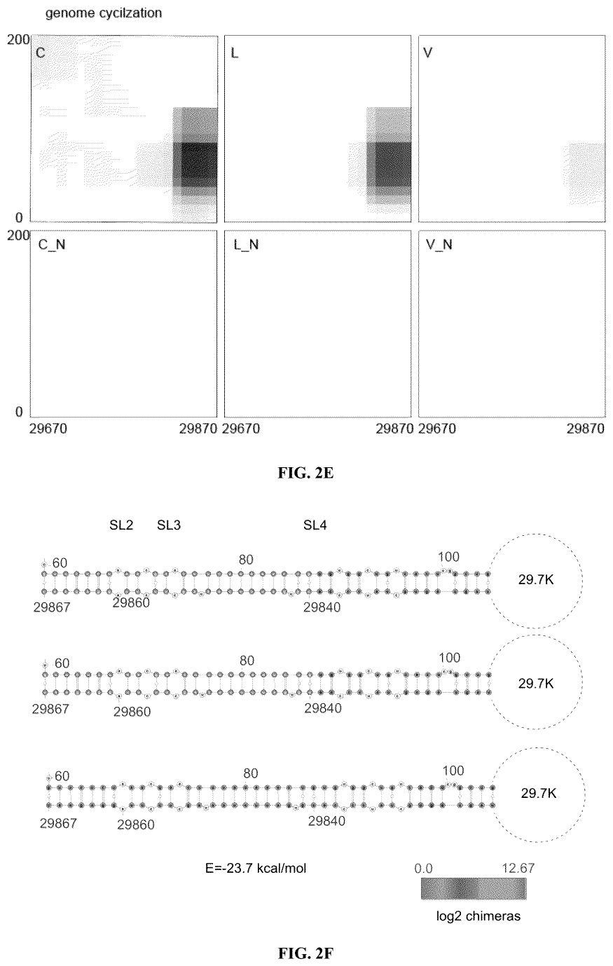

### DETAILED DESCRIPTION OF THE EMBODIMENTS

The present disclosure provides a PLA-based detection method for HOS of an RNA virus, which includes the following steps:

1) mixing an RNA virus with a cross-linking agent, conducting cross-linking under UV light, and recovering the RNA virus to obtain a cross-linked RNA virus;

2) extracting RNA of the cross-linked RNA virus in step 1);

3) conducting fragmentation on the RNA in step 2) with RNase III to obtain RNA fragments;

4) ligating the RNA fragments in step 3) and conducting decrosslinking to obtain decrosslinked RNA fragments;

5) constructing a sequencing library for the decrosslinked RNA fragments in step 4); and

6) conducting high-throughput sequencing on the sequencing library in step 5), and conducting an RNA HOS analysis on a sequencing result.

In the present disclosure, the RNA virus is mixed with the cross-linking agent, cross-linked under the UV light, and the RNA virus is recovered to obtain the cross-linked RNA virus.

The method is applicable to all types of RNA viruses. In an example of the present disclosure, a specific implementation method is illustrated by taking the coronavirus and the Coxsackie virus as examples.

In the present disclosure, a preparation method of the RNA virus preferably includes infecting cells with an RNA virus, culturing and isolating the RNA virus to obtain RNA virus particles. The infecting is preferably conducted for 20-25 h, more preferably 24 h. The RNA virus has a multiplicity of infection (MOI) of 0.01. The cells have a concentration of 1.0×10⁷-1.0×10⁹/ml.

In the present disclosure, the collected RNA virus mixed with the cross-linking agent has a final concentration of preferably 10⁷-10⁹ copies/mL, more preferably 5×10⁷-5×10⁸ copies/mL. A mixed system has a total volume of preferably 50 μl to 10 ml, more preferably 100 μl. The cross-linking agent is preferably a PBS solution containing a psoralen-derived cross-linking agent. The psoralen-derived cross-linking agent has a final concentration of preferably 1-4 μmol/L, more preferably 2 μmol/L. The psoralen-based cross-linking agent preferably includes AMT or EZ-Link™ Psoralen-PEG3-Biotin. The cross-linking agent preferably further includes digitonin; the digitonin has a mass concentration of preferably 0.01-1%, more preferably 0.01-0.5%. The digitonin as a penetrating agent promotes penetration of the cross-linking agent to reach the RNA through viral capsid protein to improve the cross-linking efficiency.

In the present disclosure, the UV light has a wavelength of preferably 360-370 nm, more preferably 365 nm. The cross-linking is preferably conducted in an ice bath for preferably 5-25 min, more preferably 10-20 min, most preferably 12 min. The cross-linking by UV light facilitates the formation of covalent bonds between the RNA molecules interacting in the virus, and provides convenience for subsequent proximity ligation reaction.

In the present disclosure, there is no specific limitation on a recovery method of the RNA virus, and recovery methods well known in the art can be employed.

In the present disclosure, the RNA of the cross-linked RNA virus is extracted after obtaining the cross-linked RNA virus.

In the present disclosure, there is no specific limitation on the extraction method of the RNA virus, and extraction methods well known in the art can be employed, such as a Trizol method or RNA extraction by an RNeasy Plus Mini Kit (Qiagen).

In the present disclosure, quantitative detection and mass detection of the extracted RNA were preferably conducted. The quantitative detection preferably detects the concentration of the RNA using Qubit to provide guidance for subsequent sampling volume. RNA integrity is preferably detected using Agilent 2100, with a recommended RIN value of greater than 7.

In the present disclosure, the RNA from the RNA virus is preferably detected for UV cross-linking effect. The UV cross-linking effect is preferably detected using a Dotblot kit.

In the present disclosure, fragmentation is conducted on the RNA with RNase III to obtain RNA fragments.

In the present disclosure, a reaction system for the fragmentation with RNase III in step 3) preferably includes 1 μl of 10×RNase III buffer, 200 ng of RNA and 1 μl of RNase III, supplemented to 20 μl with RNase-free water. The fragmentation with RNase III is conducted for preferably 1-10 min, more preferably 2-8 min, and further more preferably 5 min at preferably 36-38° C., more preferably 37° C. The RNA fragments obtained using fragmentation with RNase III can be directly ligated; while RNA fragments obtained using fragmentation with other types of endonucleases, terminals of the RNA fragments need to be subjected to polynucleotide kinase (PNK) treatment before ligation. Therefore, the RNase III enzyme can reduce the experimental steps, reduce the loss of RNA during the experiment and improve the reaction efficiency.

In the present disclosure, the obtained RNA fragments are ligated and decrosslinked to obtain decrosslinked RNA fragments.

In the present disclosure, a reaction system of the ligation includes 20 μl of 10×T4 RNA Ligase buffer, 20 μl of 10 Mm ATP, 1 μl of Superase In, 5 μl of Ribolock RI, 5 μl of T4 RNA Ligase 1 and 200 ng of RNA fragments, supplementing to 200 μl with RNase-free water. The ligation is preferably conducted in a water bath at 16° C. overnight.

The ligated RNA fragments are preferably purified after decrosslinking. In the present disclosure, there is no specific limitation on a purification method, and purification methods well known in the art can be employed, such as recovering trace amounts of RNA using RNeasy Plus Mini Kit RNA (Qiagen).

In the present disclosure, the decrosslinking is preferably conducted by irradiating the RNA fragments with UV light. The UV light preferably has a wavelength of 250-260 nm, more preferably 254 nm. The irradiating preferably lasts for 1-10 min, more preferably 5 min. The cross-linking is preferably conducted in an ice bath. The purpose of the decrosslinking is to destroy the covalent bond to avoid the covalent bond formed by the crosslinking in subsequent library construction from affecting the reverse transcription reaction.

In the present disclosure, a sequencing library is constructed for the decrosslinked RNA fragments.

In the present disclosure, the decrosslinked RNA fragments are preferably detected using Agilent 2100 before sequencing library construction. In the present disclosure, there is no specific limitation on a sequencing library construction method, and sequencing library construction methods well known in the art can be employed, such as SMARTer Stranded Total RNA-Seq Kit v2-Pico Input Mammalian User Manual.

In the present disclosure, high-throughput sequencing is conducted on the sequencing library, and an RNA HOS analysis is conducted on a sequencing result.

In the present disclosure, there is no specific limitation on a sequencing method of the high-throughput library, and sequencing methods of the high-throughput library well known in the art can be employed. In the present disclosure, the high-throughput sequencing is completed by entrusting Annoroad Gene Technology Co., Ltd.

In the present disclosure, a chimeric read analysis of the sequencing results preferably refers to the prior art (Travis, A. J., Moody, J., Helwak, A., Tollervey, D., & Kudla, G. (2014)). Hyb: a bioinformatics pipeline for the analysis of CLASH (crosslinking, ligation and sequencing of hybrids) data. Methods, 65(3), 263-273. Doi:10.1016/j.ymeth.2013.10.015).

In the present disclosure, the method can analyze the HOS of an RNA viral genome in supernatant virus particles obtained by cell culture or collection using a high-efficiency proximity ligation. In addition, the method conducts experiments using total RNA initial amount as low as 200 ng, which meets requirements for RNA structural study. Therefore, the method provided by the present disclosure can greatly improve the applicability of the proximity ligation in studies of a structure of microRNAs such as viruses.

For further description, the PLA-based detection method for HOS of an RNA virus provided by the present disclosure is described in detail below with reference to the examples, but these examples may not be understood as limiting the protection scope of the present disclosure.

### Example 1

Application of Hi-R Technology in Analysis of COVID-19 Genome Structure

1. Experimental materials were as follows: Vero cells infected with new coronavirus (SARS-CoV-2), supernatant; cross-linking agent: EZ-Link Psoralen-PEG3-Biotin (Thermo Fisher Scientific) and Permeabilizing agent: digitonin (Sigma-Aldrich).

2. Experimental Steps

2.1. Cross-Linking

9×10⁷/ml of VeroE6 was infected with Wuhan-Hu-1 SARS-CoV-2 virus in a MOI of 0.01 for 24 hours. Three duplicate samples were washed with PBS three times, and the washed cells were collected (denoted as C1, C2 and C3). Remaining infected samples were continued to be cultured for 48 hours, and a virus culture supernatant was mixed with an equal volume of a saturated sodium sulfate solution for 1 hour at 4° C. A mixture was washed with PBS three times and above virus pellets (denoted as V1, V2 and V3) and washed cells (denoted as L1, L2 and L3) were collected. EZ-Link Psoralen-PEG3-Biotin was diluted to 2 μM in a PBS containing 0.01% digitonin, and virus particles or cells were resuspended. After incubating at 37° C. for 10 minutes, the virus particles or cells were spread evenly into one well of a 6-well plate. The 6-well plate was put into a cross-linker after removing cover of the of plate, and cross-linked twice for 10 minutes under 365 nm (the cross-linking machine was placed in a safety cabinet). The 6-well plate was placed on ice during each run of cross-linking. After 10 minutes of cross-linking, the 6-well plate was taken out for new ice replacement, and the cross-linking was continued.

2.2. Extraction of RNA

RNeasy mini kit (Qiagen) was used follow the kit instructions.

2.3. RNA Fragmentation

An RNA fragmentation reaction system was prepared as shown in Table 1 for details.

TABLE 1
RNA fragmentation reaction system

| | Volume (μl) | Remarks |
|---|---|---|
| RNase III buffer (10×) | 1 | |
| RNA | — | 200 ng |
| RNase-free water | | Supplemented to 20 μl |
| RNase III | 1 | |

The reaction system was incubated at 37° C. for 5 minutes, and immediately transferred to RNA purification.

2.4. Purification of Fragmented RNA

Trace RNA was recovered with RNeasy Plus Mini Kit RNA (Qiagen), following the instructions for use.

2.5. Ligation

A ligation system was prepared using agents as shown in Table 2.

TABLE 2
Ligation system

| | Volume (μl) | Remarks |
|---|---|---|
| T4 RNA Ligase buffer (10×) | 20 | |
| 10 Mm ATP | 20 | |
| Superase In | 1 | |
| Ribolock RI | 5 | |
| T4 RNA Ligase 1 | 5 | |
| RNA | — | (200 ng) |
| RNase-free water | | Supplemented to 200 μl |

The reaction system was mixed evenly in a 16° C. water bath overnight.

2.6. Purification of Ligated RNA

Trace ligated RNA was recovered with RNeasy Plus Mini Kit RNA, following the instructions for use.

2.7. Decrosslinking

The cover of RNase-free EP tube was cut off on an ultra-clean workbench, and the recovered RNA was added to the RNase-free EP tube cover and subjected to 254 nm UV radiation on the ice for 5 min to decrosslink.

2.8. Construction of Sequencing Library

Before library construction, the RNA was detected using Agilent 2100. The library construction referred to SMARTer Stranded Total RNA-Seq Kit v2-Pico Input Mammalian User Manual.

2.9. High-Throughput Sequencing and HOS Analysis

The sequencing was conducted with Novaseq 6000, and the sequencing library was provided according to the requirements of a sequencing service provider. The sequencing results was compared with the prior art (Travis, A. J., Moody, J., Helwak, A., Tollervey, D., & Kudla, G. (2014)). Hyb: a bioinformatics pipeline for the analysis of CLASH (crosslinking, ligation and sequencing of hybrids) data. Methods, 65(3), 263-273. Doi:10.1016/j.ymeth.2013.10.015); and RNA HOS analysis of the COVID-19 was conducted.

3. Experimental Results

3.1. Evaluation of Sample Data of Each Group

Results of evaluation of sample data of each group were shown in Table 3.

TABLE 3
Evaluation of sample data of each group

| | | Deduplication | Single | Chimeric | Chimeric |
|---|---|---|---|---|---|
| | | sequencing | fragment | fragment | fragment |
| Sample | Sample type | amount | amount | amount | ratio |
| C1 | Cell cross-linking and ligation | 8135221 | 7144691 | 990530 | 0.121758217 |
| | in early infection, litigated | | | | |
| C2 | Cell cross-linking and ligation | 8118373 | 7050580 | 1067793 | 0.131527955 |
| | in early infection, litigated | | | | |
| C3 | Cell cross-linking and ligation | 6699934 | 5898326 | 801608 | 0.119644164 |
| | in early infection, litigated | | | | |
| L1 | Cell cross-linking and ligation in | 11147030 | 10063584 | 1083446 | 0.097195935 |
| | late infection, litigated | | | | |
| L2 | Cell cross-linking and ligation in | 9608398 | 8642304 | 966094 | 0.100546834 |
| | late infection, litigated | | | | |
| L3 | Cell cross-linking and ligation in | 7651263 | 6604034 | 1047229 | 0.136870083 |
| | late infection, litigated | | | | |
| V1 | Cross-linking and ligation of | 17048301 | 12836864 | 4211437 | 0.247029719 |
| | virus supernatant, litigated | | | | |
| V2 | Cross-linking and ligation of | 17667641 | 13288720 | 4378921 | 0.247849784 |
| | virus supernatant, litigated | | | | |
| V3 | Cross-linking and ligation of | 12145848 | 8626630 | 3519218 | 0.289746587 |
| | virus supernatant, litigated | | | | |
| C1-N | Cell cross-linking and no | 4609243 | 4532456 | 76787 | 0.016659352 |
| | ligation in late infection, | | | | |
| | non-litigated | | | | |
| L1-N | Cell cross-linking and no | 6066508 | 5959962 | 106546 | 0.017562987 |
| | ligation in late infection, | | | | |
| | non-litigated | | | | |
| V1-N | Cross-linking and no ligation of | 18299077 | 18218860 | 80217 | 0.004383664 |
| | virus supernatant, non-litigated | | | | |

It can be seen from the above data that the proportion of chimeric fragments in the ligation group is significantly higher than that of a non-ligated control group, and the proportion of chimeric fragments in the ligation group of infected cells is around 10%. The proportion of chimeric fragments ligated to the virus supernatant exceeds 20%, indicating that the RNA compression is relatively tight.

As a control, the ligation efficiency of the COVID-19 genome structure detected using a similar COMRADES method was further analyzed. Results are shown in Table 4.

TABLE 4
Results of detection of COVID-19 genome structure by
the method of this example and the COMRADES method

| | | | Deduplication | Single | Chimeric | |
|---|---|---|---|---|---|---|
| | | | Sequencing | fragment | fragment | Chimeric |
| Sample | Sample type | Ligation | volume | amount | amount | fragment ratio |
| SRR12252273 | COVID-19 | − | 30954420 | 30594439 | 359981 | 0.011629389 |
| | gRNA | | | | | |
| SRR12252274 | COVID-19 | + | 22837576 | 21425685 | 1411891 | 0.061823155 |
| | gRNA | | | | | |
| SRR12252275 | COVID-19 | − | 33861701 | 33450236 | 411465 | 0.012151339 |
| | gRNA | | | | | |
| SRR12252281 | COVID-19 | − | 45719034 | 45196124 | 522910 | 0.011437468 |
| | sgRNA | | | | | |
| SRR12252282 | COVID-19 | + | 20939249 | 20298523 | 640726 | 0.030599283 |
| | sgRNA | | | | | |
| SRR12252283 | COVID-19 | − | 51181530 | 50545553 | 635977 | 0.012425908 |
| | sgRNA | | | | | |
| SRR12252284 | COVID-19 | + | 60754387 | 58849116 | 1905271 | 0.031360221 |
| | sgRNA | | | | | |

Determining from the overall detection results in Table 3 and Table 4, the method provided by the present disclosure increases the ratio of chimeric fragments produced by ligation, that is, the effective data ratio is increased. The main reason may be that all terminals are suitable for ligation after RNase III fragmentation to greatly improve the efficiency of ligation.

Meanwhile, in this example, by analyzing the structure of the COVID-19 genome at different life stages, data analysis shows the reliability of the technology, and the details of the internal interaction of the COVID-19 genome can be found. The mechanism of COVID-19 transcription is revealed by analyzing TRS-L-mediated interactions. The similarities and differences of the COVID-19 genome structure in different life states are compared in the details of interaction and the overall structural domain of the genome. Details are as follows.

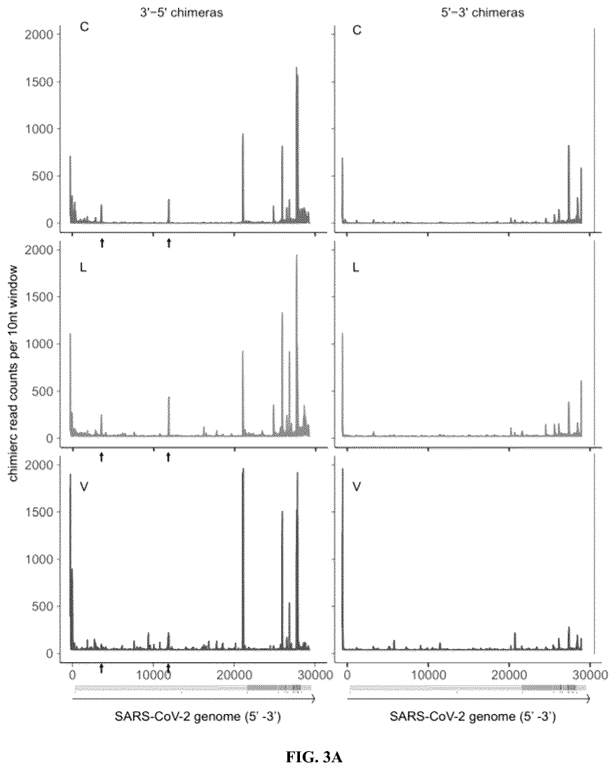

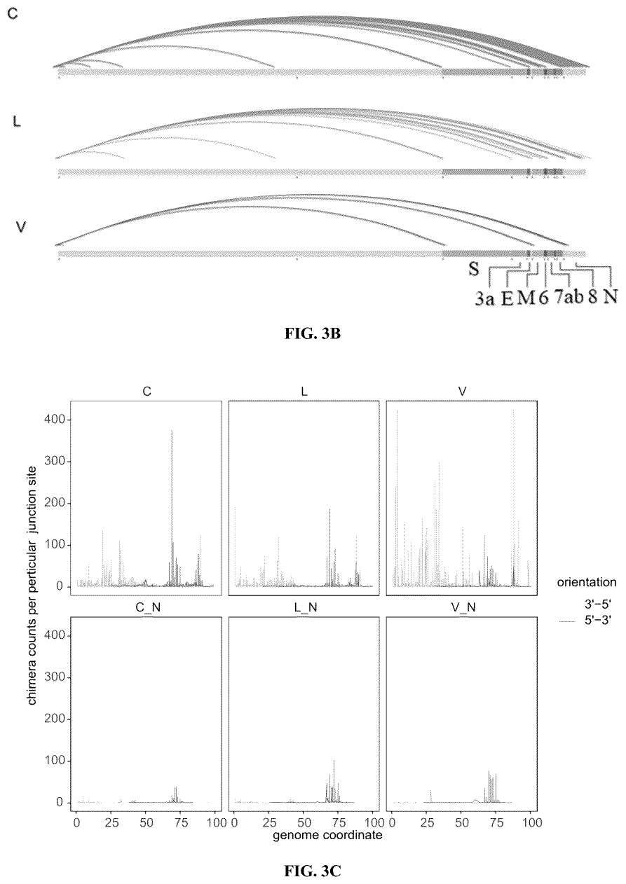

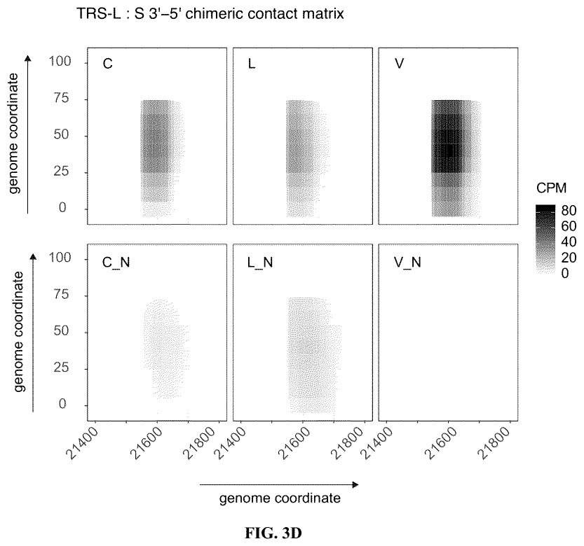

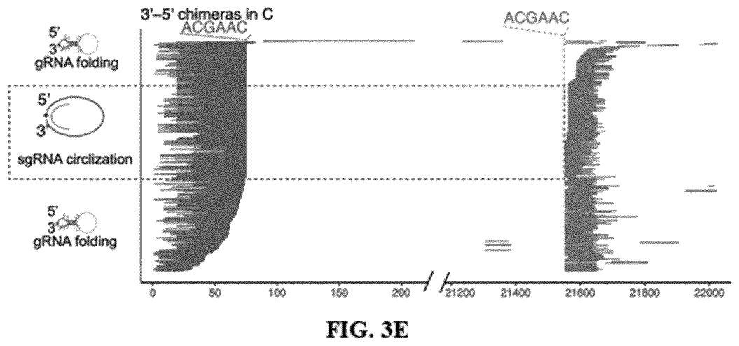

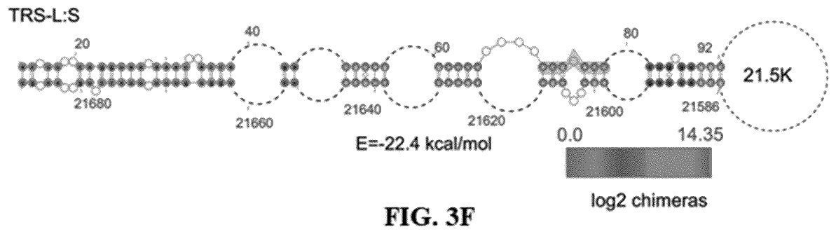

FIG. 3 shows the remote interaction results of the TRS-L seat and the TRS-B seat discovered by the Hi-R method. The results in FIG. 3 show that the high-throughput sequencing data generated by this technology can be used to reveal the details of long-distance interactions closely related to the transcription of the COVID-19.

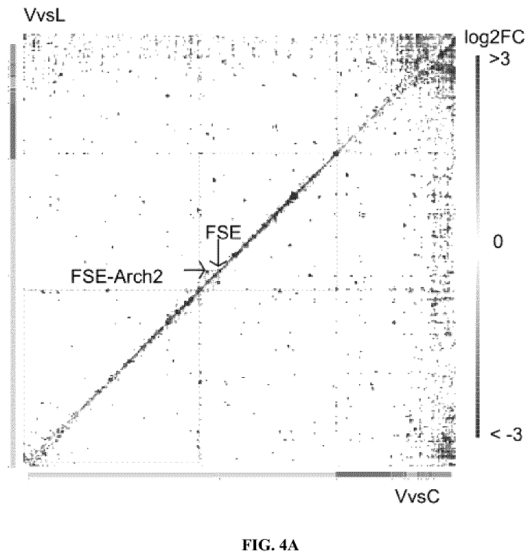

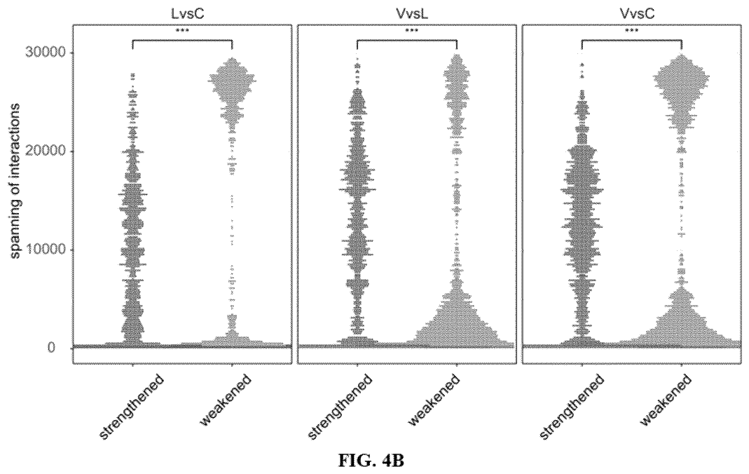

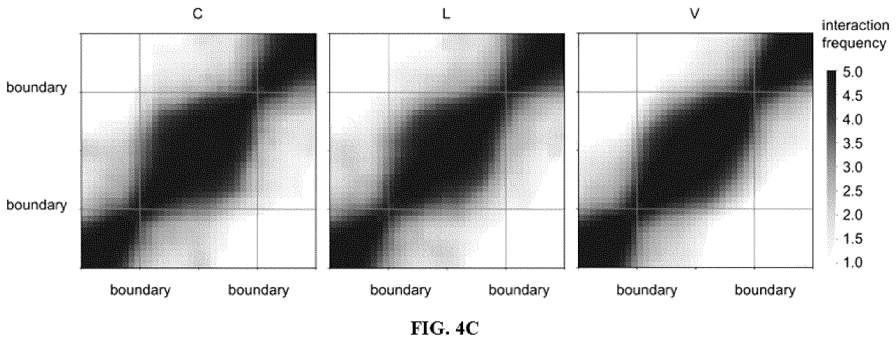

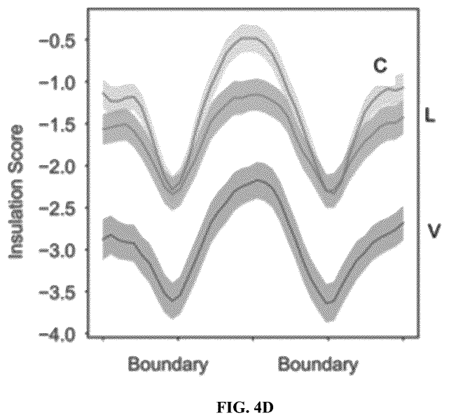

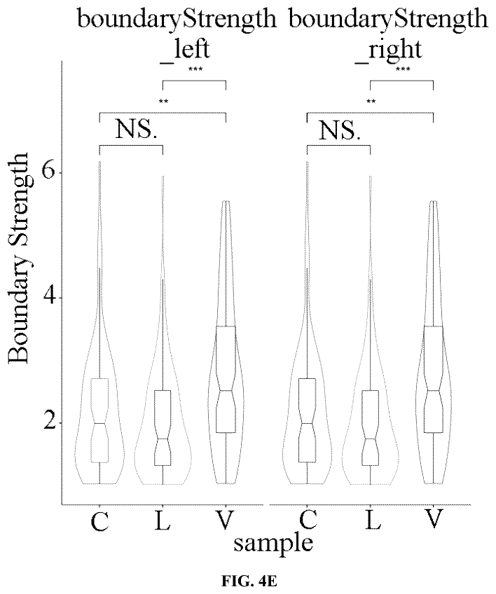

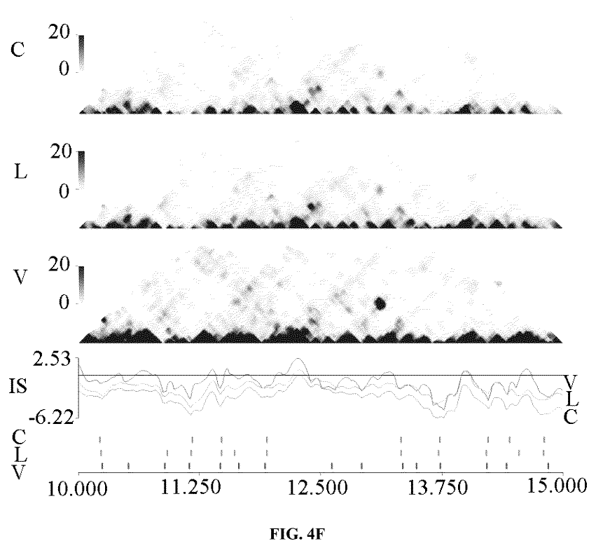

FIG. 4 shows the comparison results of structures of a virus in different states. FIG. 4A is a heat map showing the comparison of RNA-RNA interaction in virus particles and cells of the early stage of infection (VvsC) and in virus particles and cell lysates of the late stage of infection (VvsL), where the VvsL is in the upper quadrant and the VvsC is in the lower quadrant. FIG. 4B shows a span distribution of the interaction with different intensities, where a dot plot shows a distribution of differential interaction, *** p<0.001, and two-way and two-sample Kolmogorov-Smirnov test. FIG. 4C shows that the structural domain characteristics are maintained during the life cycle of the SARS-CoV-2 virus, and it is deduced that the interaction frequency within the close-range domains presented by the interaction within the genome is higher than the interaction characteristics between the domains; where a heat map shows the normalized average interaction frequency of all boundaries and their vicinity (±0.5 domain length) in the C, L and V samples, and the heat map is divided into windows with a resolution of 10 nt. FIG. 4D maps an average normalized insulation score around the boundary from ½ upstream to ½ downstream. FIG. 4E is a violin diagram comparing the boundary strength of the C, L and V samples, showing a higher boundary strength in the V sample. FIG. 4F is an RNA interaction map (top) aliquoted with a resolution of 10 nt, showing that an interaction distance on the SARS-CoV-2 genome in the C, L and V samples is 10-15 kb, where a line graph (median) shows an insulation curve, and the short line (bottom) reflects the boundaries. In summary, FIG. 4 shows that the high-throughput sequencing data generated by the method of the present disclosure can explain the rules of the folding structure of the COVID-19 genome, and compare the dynamic characteristics of the folding of the virus in different life states.

### Example 2

Application of Hi-R Technology in Analysis of Coxsackie Viral Genome Structure

1. Experimental Materials

Virus particles from the supernatant of HeLa cells infected with Coxsackie virus (CVB-3).

Cross-linking agent: EZ-Link Psoralen-PEG3-Biotin (Thermo Fisher Scientific).

Permeabilizing Agent: Digitonin (Sigma-Aldrich)

2. Experimental Steps

2.1. Crosslinking

1×10⁸/ml of HeLa cells were infected with CVB-3 strain in a MOI of 0.01 for 24 hours. The virus was concentrated by ultracentrifugation. Concentrated virus was filtered with 0.6-μm microporous membrane, transferred to a 38-ml ultrafiltration centrifuge tube, 5 ml of 35% sucrose solution filtered through a 0.2-μm microporous membrane was carefully added to the bottom of the ultrafiltration centrifuge tube. Opening was sealed with soldering iron. Virus particles were centrifuged at 4° C. and 100,000 g for 16 h to the bottom of the tube, upper-layer medium was carefully removed, and the virus particles were collected. The virus particles was resuspended with 100 μl of 2 μM cross-linking agent (containing 0.1% Permeabilizing agent), and incubated at 37° C. for 10 min. The virus particles were spread evenly into one well of a 6-well plate. The 6-well plate was put into a cross-linking machine after removing a cover, and cross-linked twice for 10 minutes at 365 nm. It was required that the cross-linker was placed in a safety cabinet. The 6-well plate was placed on ice during each run of cross-linking. After 10 minutes of cross-linking, the 6-well plate was taken out for replacement of new ice, and the cross-linking was continued. Following the cross-linking, the 6-well plate was taken out and cross-linked virus was treated with 1 ml of Trizol. RNA was extracted using a Trizol method, following the instructions for use.

2.2. Extraction of RNA

RNeasy mini kit (Qiagen) was used following the kit instructions for use.

2.3. RNA Fragmentation

An RNA fragmentation reaction system was prepared using agents as shown in Table 5.

TABLE 5
RNA fragmentation reaction system

| | Volume (μl) | Remarks |
|---|---|---|
| RNase III buffer (10×) | 1 | |
| RNA | — | 200 ng |
| RNase-free water | | Supplemented to 20 μl |
| RNase III | 1 | |

The reaction system was incubated at 37° C. for 5 minutes and immediately transferred to RNA purification.

2.4. Purification of Fragmented RNA

Trace RNA was recovered with RNeasy Plus Mini Kit RNA (Qiagen), following the instructions for use.

2.5. Ligation

A ligation system was prepared using agents as shown in Table 6.

TABLE 6
Ligation system

| | Volume (μl) | Remarks |
|---|---|---|
| T4 RNA Ligase buffer (10×) | 20 | |
| 10 Mm ATP | 20 | |
| Superase In | 1 | |
| Ribolock RI | 5 | |
| T4 RNA Ligase 1 | 5 | |
| RNA | — | (200 ng) |
| RNase-free water | | Supplemented to 200 μl |

The ligation system was mixed evenly in a 16° C. water bath overnight.

2.6. Purification of Ligated RNA

30 μl Magic Pure RNA Beads (TransGen Biotech)+370 μl of Crowd buffer were added to the ligation system, and mixed well and recovered. Elution was conducted with 15 μl of RNase-free water (Note: if there are too few RNA Beads in this step, the system will be large to affect the adsorption of magnetic beads, and the purification will be very slow). Qubit quantitative analysis was conducted.

2.7. Decrosslinking

The cover of RNase-free EP tube was cut off on an ultra-clean workbench, and the recovered RNA was added to the RNase-free EP tube cover and subjected to 254 nm UV radiation on the ice for 5 min to decrosslink.

2.8. Construction of Sequencing Library

Before library construction, the RNA was detected using Agilent 2100. The library construction referred to SMARTer Stranded Total RNA-Seq Kit v2-Pico Input Mammalian User Manual.

2.9. High-Throughput Sequencing

The sequencing was conducted by Hiseq Xten, and the high-throughput sequencing was entrusted to be completed by Annoroad Gene Technology Co., Ltd.

3. Experimental results are shown in Table 7.

TABLE 7
Detection results of the method of this example for detecting Coxsackie virus

| | | | Deduplication | Single | Chimeric | |
|---|---|---|---|---|---|---|
| | | | sequencing | fragment | fragment | Chimeric |
| Sample | Virus type | Ligation | volume | amount | amount | fragment ratio |
| Csv2 | GFP insertion | − | 495039 | 486943 | 8096 | 0.016626176 |
| Csv3 | GFP insertion | + | 2480669 | 2185149 | 295520 | 0.135240206 |
| Csv4 | GFP insertion | + | 2010946 | 1765974 | 244972 | 0.138717784 |
| CVB31 | Wild type | + | 6663009 | 5647358 | 1015651 | 0.179845337 |
| CVB32 | Wild type | + | 7189921 | 6127418 | 1062503 | 0.173401423 |
| CVB41 | Wild type | + | 5570237 | 4619109 | 951128 | 0.205911573 |
| CVB42 | Wild type | + | 5406942 | 4495926 | 911016 | 0.202631449 |

The results in Table 7 show that the proportion of chimeric fragments after ligation is much higher than that in the non-ligated group.

The data obtained by sequencing more intuitively shows that the Hi-R technology proposed by the present disclosure can reveal the genomic structural characteristics of the Coxsackie virus CVB13 type, and can be used to compare the structures of two strains of viruses. That is, the intensity of the interaction can be observed, and the domain characteristics of the entire genome can also compared. Details are as follows:

FIG. 5 shows results of contact matrix comparing two biological duplicates, where a Coxsackie virus particle RNA of the two biological replicates was processed by Hi-R experiment, a contact matrix diagram shows that the biological replicates have high similarity.

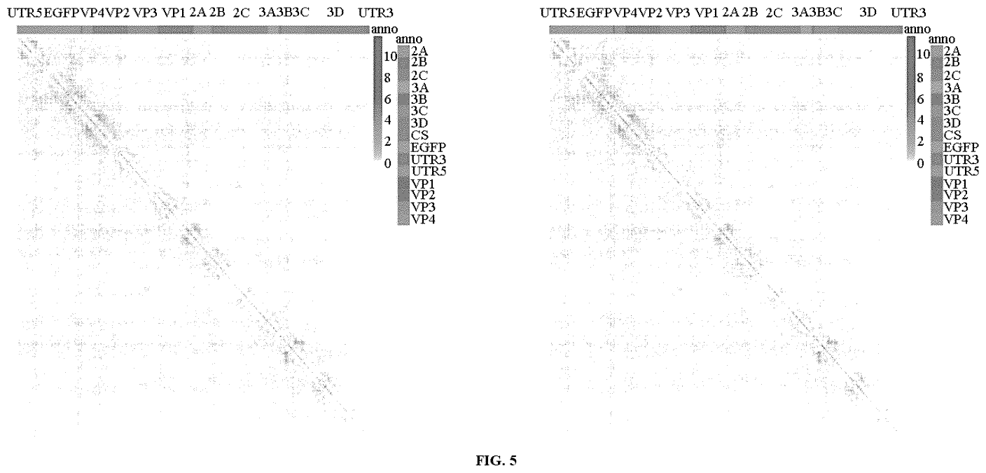

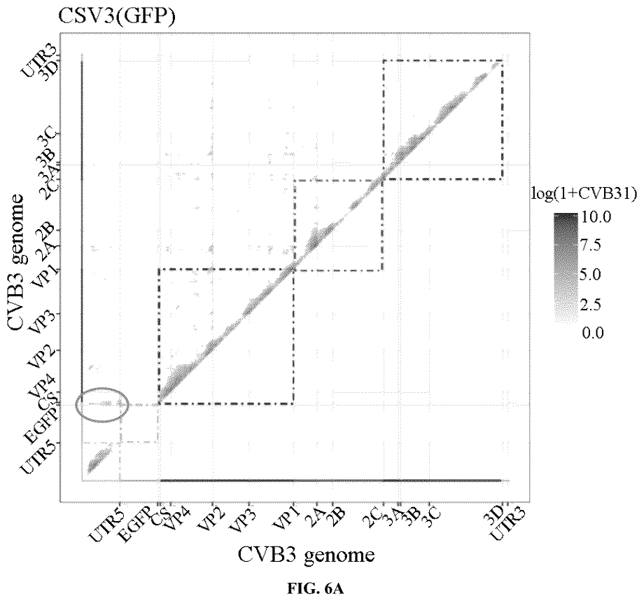

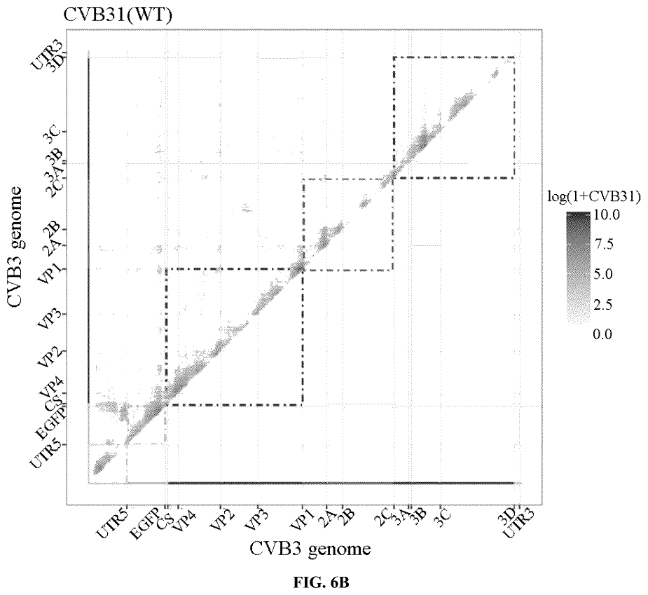

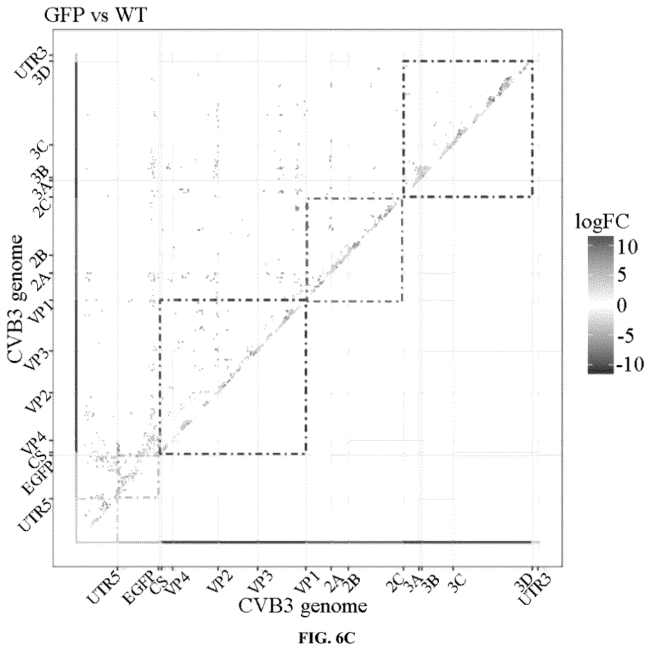

FIG. 6 is a comparison result of Coxsackie virus structure before and after GFP insertion. FIG. 6A is a heat map of RNA-RNA interaction of a Coxsackie virus CVB3 type. FIG. 6B is a heat map of RNA-RNA interaction of the Coxsackie virus CVB3 type after GFP insertion. FIG. 6C is a difference map of interaction before and after GFP insertion, where red dots represent enhanced interactions after GFP insertion, and blue dots represent weakened interactions after GFP insertion. It can be seen from FIG. 6 that the high-throughput sequencing data obtained by the method of the present disclosure can reveal the changes of the Coxsackie virus fragment interaction before and after transformation.

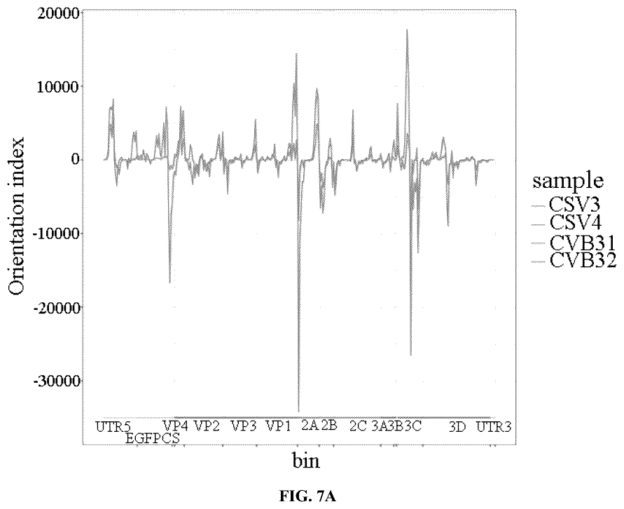

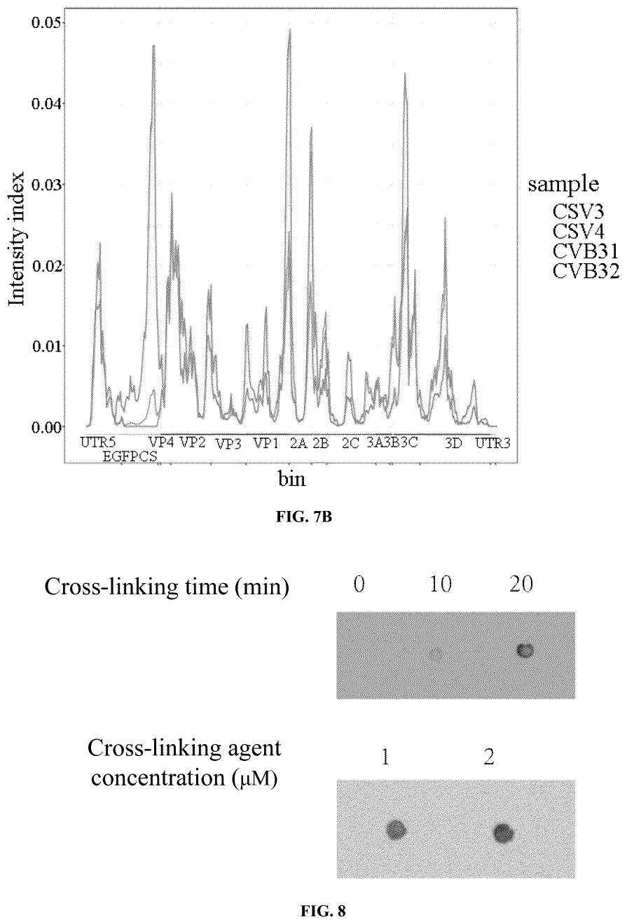

FIG. 7 shows results of comparing structural characteristics of two Coxsackie viruses. FIG. 7A shows characteristics of a Coxsackie viral genome domain before and after GFP insertion described using orientation index, showing that the domain is enhanced after GFP insertion. FIG. 7B shows characteristics of the Coxsackie viral genome domain before and after GFP insertion described using intensity index, showing that the domain is enhanced after GFP insertion. It can be seen from FIG. 7 that the high-throughput sequencing data obtained by the method of the present disclosure can reveal the changes of the Coxsackie virus folding domain before and after transformation.

### Example 3

The cross-linking efficiency of Coxsackie virus RNA was determined using a Dotplot method, and the method specifically included following steps: a certain concentration (1 μM or 2 μM) of PBS (containing 0.01% digitonin) of EZ-Link Psoralen-PEG3-Biotin was mixed with a sample of Coxsackie virus particles, and cross-linked under 365 nm of UV light for different time (0, 10 min and 20 min), biotin signals were detected in the sample; a thicker spot indicated a higher cross-linking efficiency. For the Dotplot method, reference can be made to the prior art (Aw, JG, Shen, Y., Wilm, A., Sun, M., Lim, X N, Boon, K L. Wan, Y. (2016). In Vivo Mapping of Eukaryotic RNA Interactomes Reveals Principles of Higher-Order Organization and Regulation. Mol Cell, 62(4), 603-617. doi:10.1016/j.molcel.2016.04.028).

The results are shown in FIG. 8. The upper panel in FIG. 8 shows the biotin signal intensity of EZ-Link™ Psoralen-PEG3-Biotin cross-linking agent with a final concentration of 2 μM at different cross-linking time points, suggesting that the cross-linking efficiency of 20 min is better than that of 10 min. The lower panel in FIG. 8 shows cross-linking effects of 1 μM and 2 μM of cross-linking agents, respectively. Both 1 μM and 2 μM of cross-linking agents can achieve relatively ideal cross-linking efficiency. Compared with cross-linking concentration in 1 μM, cross-linking concentration in 2 μM has a better cross-linking efficiency.

The above description of examples is merely provided to help understand the method of the present disclosure and a core idea thereof. It should be noted that several improvements and modifications may be made by persons of ordinary skill in the art without departing from the principle of the present disclosure, and these improvements and modifications should also fall within the protection scope of the present disclosure. Various amendments to these embodiments are apparent to those of professional skill in the art, and the general principles defined herein may be implemented in other embodiments without departing from the spirit or scope of the present disclosure. Thus, the present disclosure is not limited to the examples shown herein but falls within the widest scope consistent with the principles and novel features disclosed herein.

What is claimed is:

1. A method for constructing a sequencing library for proximity ligation assay-based detection of a high-order structure of an RNA virus, comprising the following steps:
1) mixing an RNA virus with a cross-linking agent, conducting cross-linking under ultraviolet light, and recovering the RNA virus to obtain a cross-linked RNA virus;
2) extracting RNA of the cross-linked RNA virus in step 1);
3) conducting fragmentation on the RNA extracted in step 2) with RNase III to obtain RNA fragments;
4) ligating RNA fragments in step 3) and conducting decrosslinking to obtain decrosslinked RNA fragments; and
5) constructing a sequencing library for the decrosslinked RNA fragments in step 4).

2. The method according to claim 1, wherein the cross-linking agent in step 1) is a phosphate-buffered saline (PBS) solution containing a psoralen-derived cross-linking agent; and
the psoralen-derived cross-linking agent has a final concentration of 1-4 μmol/L.

3. The method according to claim 2, wherein the psoralen-based cross-linking agent comprises 4′-aminomethyl-4,5′,8-trimethylpsoralen (AMT) or EZ-Link™ Psoralen-PEG3-Biotin.

4. The method according to claim 2, wherein the cross-linking agent further comprises digitonin with a mass concentration of 0.01-1%.

5. The method according to claim 1, wherein the RNA virus has a final concentration of 10⁷-10⁹ copies/mL after the mixing in step 1).

6. The method according to claim 1, wherein in step 1), the UV light has a wavelength of 360-370 nm; and
the cross-linking lasts for 15-25 min.

7. The method according to claim 1, wherein a reaction system for the fragmentation with an RNase III in step 3) comprises 1 μl of 10×RNase III buffer, 200 ng of RNA and 1 μl of RNase III, supplemented to 20 μl with RNase-free water.

8. The method according to claim 1, wherein the fragmentation with RNase III is conducted for 1-10 min at 36-38° C.

9. The method according to claim 1, wherein in step 4), the decrosslinking is conducted by irradiating the RNA fragments with the UV light;
the UV light has a wavelength of 250-260 nm; and
the irradiating lasts for 1-10 min.

10. The method according to claim 1, wherein the RNA virus comprises a coronavirus and/or a Coxsackie virus.

11. A proximity ligation assay-based method for detecting a high-order structure of an RNA virus, comprising the following steps:
1) mixing an RNA virus with a cross-linking agent, conducting cross-linking under ultraviolet (UV) light, and recovering the RNA virus to obtain a cross-linked RNA virus;
2) extracting RNA of the cross-linked RNA virus in step 1);
3) conducting fragmentation on the RNA extracted in step 2) with RNase III to obtain RNA fragments;
4) ligating the RNA fragments in step 3) and conducting decrosslinking to obtain decrosslinked RNA fragments;
5) constructing a sequencing library for the decrosslinked RNA fragments in step 4); and
6) conducting high-throughput sequencing on the sequencing library in step 5), and conducting an RNA HOS analysis on a sequencing result.

12. The proximity ligation assay-based method according to claim 11, wherein the cross-linking agent in step 1) is a phosphate-buffered saline (PBS) solution containing a psoralen-derived cross-linking agent; and
the psoralen-derived cross-linking agent has a final concentration of 1-4 μmol/L.

13. The proximity ligation assay-based method according to claim 12, wherein the psoralen-based cross-linking agent comprises 4′-aminomethyl-4,5′,8-trimethylpsoralen (AMT) or EZ-Link™ Psoralen-PEG3-Biotin.

14. The proximity ligation assay-based method according to claim 12, wherein the cross-linking agent further comprises digitonin with a mass concentration of 0.01-1%.

15. The proximity ligation assay-based method according to claim 11, wherein the RNA virus has a final concentration of 10⁷-10⁹ copies/mL after the mixing in step 1).

16. The proximity ligation assay-basedmethod according to claim 11, wherein in step 1), the UV light has a wavelength of 360-370 nm; and
the cross-linking lasts for 15-25 min.

17. The proximity ligation assay-based method according to claim 11, wherein a reaction system for the fragmentation with RNase III in step 3) comprises 1 μl of 10×RNase III buffer, 200 ng of RNA and 1 μl of RNase III, supplementing to 20 μl with RNase-free water.

18. The proximity ligation assay-based method according to claim 11, wherein the fragmentation with an RNase III is conducted for 1-10 min at 36-38° C.

19. The proximity ligation assay-based method according to claim 11, wherein in step 4), the decrosslinking is conducted by irradiating the RNA fragments with the UV light;
the UV light has a wavelength of 250-260 nm; and
the irradiating lasts for 1-10 min.

20. The proximity ligation assay-based method according to claim 11, wherein the RNA virus comprises a coronavirus and/or a Coxsackie virus.
